(19) United States
(12) Patent Application Publication
(10) Pub. No.: US 2025/0114188 A1
(43) Pub. Date: Apr. 10, 2025

(54) A DEVICE AND ITS USE IN A METHOD FOR CHANGING AN EYE COLOR

(71) Applicant: Elias JARADE, Dubai (AE)

(72) Inventor: Elias JARADE, Dubai (AE)

(21) Appl. No.: 18/833,251

(22) PCT Filed: Jan. 25, 2023
(86) PCT No.: PCT/IB2023/050630

§ 371 Date: Jul. 25, 2024

Related U.S. Application Data
(60) Provisional application No. 63/303,522, filed on Jan. 27, 2022.
(60) Provisional application No. 63/440,928, filed on Jan. 25, 2023.

(51) Int. Cl. A61F 2/14; A61F 2/00

(52) U.S. Cl. CPC A61F 2/142; A61F 2/0059

(57) ABSTRACT

A device and its use in a method for changing an eye color is disclosed. The device comprises a plurality of colored intracorneal ring segments of pre-defined length, width and positioning. The method comprises forming a pair of concentric intracorneal tunnels in a mid-periphery of a cornea. The pair of concentric intracorneal tunnels comprises an inner intracorneal tunnel and an outer intracorneal tunnel. The inner intracorneal tunnel is configured to accommodate a pair of colored inner ring segments wherein each of the colored inner ring segment comprises a plurality of first fenestrations. The outer intracorneal tunnel is configured to accommodate a pair of colored outer ring segments wherein, each of the colored outer ring segment comprises a plurality of second fenestrations. Furthermore, a cross-section of the pair of colored inner ring segments and the pair of colored outer ring segments is bilamellar.

### FIELD OF INVENTION

The subject matter in general relates to the field of cosmetic ophthalmology, and in particular to a method for changing eye color using reversible techniques.

### BACKGROUND

Changing eye color is highly desirable for cosmetic purposes. The eye is a highly complex, sensitive, and precious organ. The eye color is determined by the level of pigmentation within an iris of the eye. The iris is a biological tissue that contains melanin. Based on the concentration of the melanin in the iris tissue, the eye color (black, hazel, blue green, etc.) is determined. The eye color can be modified by various ways.

A conventional approach to modify the eye color involves wearing colored contact lenses. However, wearing contact lens is not desirable as it involves many complications such as but not limited to infectious keratitis and dryness.

Alternately, low energy Nd: YAG laser treatment is another approach to change the eye color. This treatment involves removal of pigment portions in the corneal stroma of the iris, using the Nd: YAG laser that results in a permanent change in the eye color. This low energy Nd: YAG laser treatment is irreversible. However, said treatment fails to precisely predict the resulting eye color post-procedure. Further, the low energy Nd: YAG laser treatment works only in one way i.e., the eye color can be changed from a darker shade to a lighter shade of the original color of the eye only and not vice versa.

In view of the foregoing discussion, there is a need for a safer, more predictable, permanent yet reversible technique to effectively change the eye color without affecting the integrity and visual acuity of the recipient's eye.

### SUMMARY

An embodiment discloses a device and its in a method for changing an eye color. The method comprises forming a pair of concentric intracorneal tunnels in a mid-periphery of a cornea. The pair of concentric intracorneal tunnels comprises an inner intracorneal tunnel and an outer intracorneal tunnel. The inner intracorneal tunnel is configured to accommodate a pair of colored inner ring segments wherein each of the colored inner ring segment comprises a plurality of first fenestrations. The outer intracorneal tunnel is configured to accommodate a pair of colored outer ring segments wherein, each of the colored outer ring segment comprises a plurality of second fenestrations. Furthermore, a cross-section of the pair of colored inner ring segments and the pair of colored outer ring segments is bilamellar.

### BRIEF DESCRIPTION OF DIAGRAMS

Exemplary embodiments of the present invention will be understood and appreciated more fully from the following detailed description, taken in conjunction with the drawings in which,

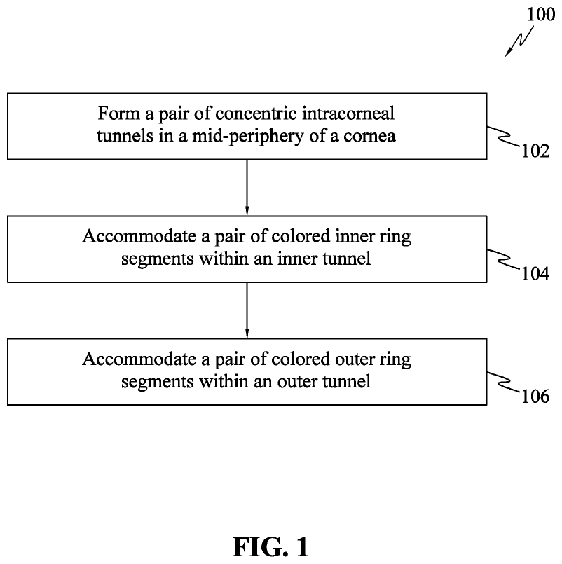

FIG. 1 illustrates a method flowchart 100 for changing an eye color, in accordance with an embodiment;

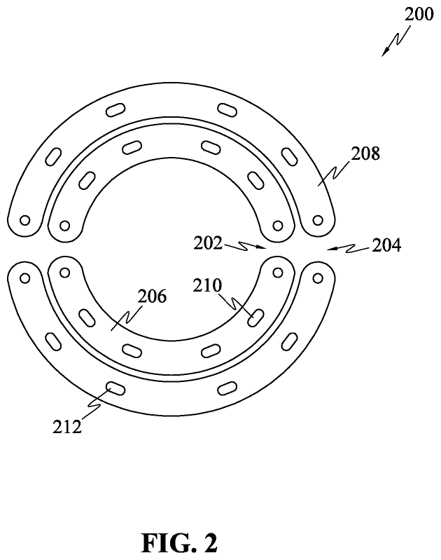

FIG. 2 illustrates a device 200, in accordance with an embodiment:

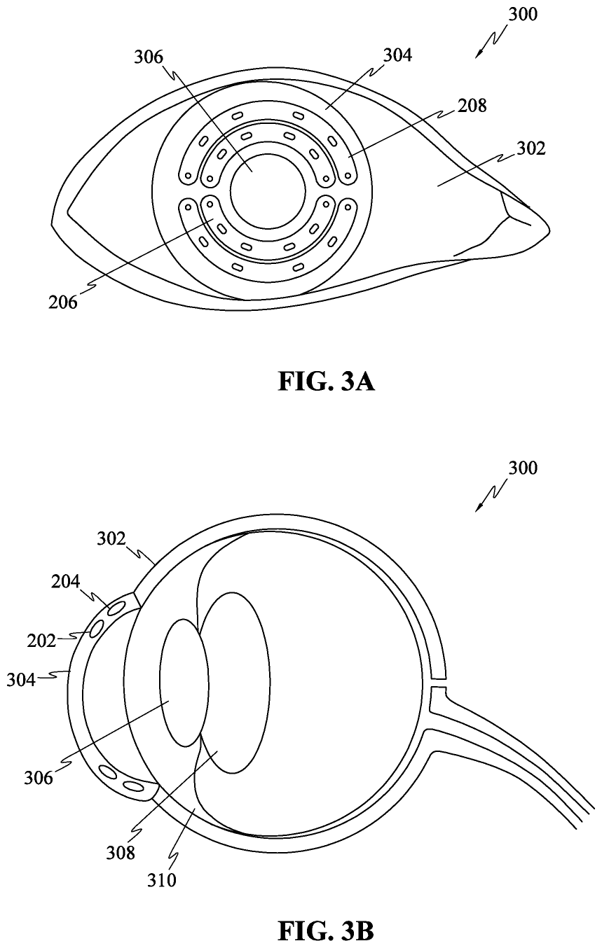

FIG. 3A illustrates a front view of an eye 300 with the device 200 implanted in a cornea 304 of the eye 300, in accordance with an embodiment; and

FIG. 3B illustrates a side view of the eye 300 with the device 200 implanted in the cornea 304 of the eye 300, in accordance with an embodiment.

### DETAILED DESCRIPTION OF THE INVENTION

The following detailed description includes references to the accompanying drawings, which form part of the detailed description. The drawings show illustrations in accordance with example embodiments. The numerals in the figure represent like elements throughout the several views, exemplary embodiments of the present disclosure are described. For convenience, only some elements of the same group may be labelled with numerals. The purpose of the drawings is to describe exemplary embodiments and not for production. Therefore, features shown in the figures are chosen for convenience and clarity of presentation only. Moreover, the language used in this disclosure has been principally selected for readability and instructional purposes and may not have been selected to delineate or circumscribe the inventive subject matter, resort to the claims being necessary to determine such inventive subject matter. These example embodiments are described in enough details to enable those skilled in the art to practice the present subject matter. However, it will be apparent to one of ordinary skill in the art that the present invention may be practiced without these specific details. The embodiments can be combined, other embodiments can be utilized, or structural and logical changes can be made without departing from the scope of the invention. The following detailed description is, therefore, not to be taken as a limiting sense.

In this document, the terms “a” or “an” are used, as is common in patent documents, to include one or more than one. In this document, the term “or” is used to refer to a non-exclusive “or,” such that “a or b” includes “a but not b,” “b but not a,” and “a and b,” unless otherwise indicated.

Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

FIG. 1 illustrates a method flow chart 100 for changing an eye color, in accordance with an embodiment. At step 102, a pair of concentric intracorneal tunnels may be formed in a mid-periphery of a cornea 304 of an eye 300 (Refer FIG. 3A). The pair of concentric intracorneal tunnels comprises an inner intracorneal tunnel 202 or inner tunnel 202, and an outer intracorneal tunnel 204 or an outer tunnel 204 (Refer FIG. 2) The pair of concentric intracorneal tunnels (202, 204) may be formed using femtosecond laser at an appropriate depth. The concentric intracorneal tunnels may be formed by any techniques that serves the purpose of the invention.

In an embodiment, the depth of each of the concentric intracorneal tunnels (202, 204) may range from 400 microns to 430 microns. The pair of concentric intracorneal tunnels (202, 204) may be configured to cover approximately an entire surface of an iris 310 of the eye 300.

FIG. 2 illustrates a device 200 comprising a pair of colored inner ring segments 206 and a pair of colored outer ring segments 208. The device 200 may be configured to be implanted in the concentric intracorneal tunnels (202, 204).

In an embodiment, the colored ring segments (206, 208) may have an arc width ranging between 800 microns to 1000 microns. Furthermore, the colored ring segments (206, 208) may have a fixed arc length of 160 degrees.

In an embodiment, a cross-section of the colored ring segments (206, 208) may be bilamellar to induce minimal corneal flattening. Further, the colored ring segments (206, 208) may have a thickness ranging between 100 microns to 200 microns.

Referring to FIG. 1, at step 104, the pair of colored inner ring segments 206 may be implanted in the inner intracorneal tunnels 202. An inner diameter of each of the pair of colored inner ring segments 206 may range between 4.0 mm to 4.5 mm. Further, an outer diameter of each of the pair of colored inner ring segments 206 may range between 6.0 mm to 6.5 mm.

In an embodiment, each of the colored inner ring segments 206 may comprise a plurality of first fenestrations 210. The plurality of first fenestrations 210 may be configured to promote normal nourishment and oxygenation of the corneal tissue in the cornea 304 of the eye 300.

At step 106, the pair of colored outer ring segments 208 may be implanted in the outer intracorneal tunnel 204. An inner diameter of each of the colored outer ring segments 208 may range between 6.8 mm to 7.0 mm. Further, an outer diameter of each of the colored outer ring segments 208 may range between 8.8 mm to 9.0 mm.

In an embodiment, each of the colored outer ring segments may comprise a plurality of second fenestrations 212. The plurality of second fenestrations 212 may be configured to promote normal nourishment and oxygenation of the corneal tissue in the cornea 304 of the eye 300.

In an embodiment, each of the colored outer ring segments 208 and inner ring segments 206 may be ultrathin to prevent altering the optical properties of the cornea. The inner and outer colored ring segments (206, 208) may be made of medical grade acrylic plastic. The medical grade acrylic plastic may be for example, but not limited to colored polymethyl methacrylate (PMMA).

In an embodiment, the implanted colored ring segments (206, 208) may be referred to as intracorneal colored ring segments (ICRS).

Referring to FIGS. 3A and 3B, the eye 300 may comprise a sclera 302, the cornea 304, a pupil 306 and the iris 310. The iris 310 may be configured to be completely covered by the pair of colored inner ring segments 206 and the pair of colored outer ring segments 208.

In an embodiment, a designated color of the ICRS may be configured to attribute the required perception of the eye color. A light traveling towards the eye 300 may enter the cornea 304, outside the inner diameter of the colored inner ring segment 206 placed in the inner intracorneal tunnel 202. The light may reflect on each of the colored inner ring segments 206 and present the color of the colored inner ring segments 206. Similarly, the light may be reflected on the surface of each of the colored outer ring segments 208 to present the color of the colored outer ring segments 208. The combination of the colors presented by the implanted device 200 may be perceived as the changed color of the eye 300.

In an embodiment, the implantation procedure of the colored ring segments 206, 208 may be reversed. The colored ring segments (206, 208) may be removed from the intracorneal tunnels (202, 204) to restore the original color of the eye 300 whenever desired. Alternately, the eye color may be changed to another color by exchanging the implanted ring segments 206 and 208 with a different colored ring segments (206, 208).

The various embodiments have been described using detailed descriptions that are provided by way of example and are not intended to limit the scope of the invention. The described embodiments comprise different features, not all of which are required in all embodiments. Some embodiments utilize only some of the features or possible combinations of the features. Many other ramification and variations are possible within the teaching of the embodiments comprising different combinations of features noted in the described embodiments.

It will be appreciated by persons skilled in the art that the various embodiments are not limited by what has been particularly shown and described herein above. Rather the scope of the invention is defined by the claims that follow.

What is claimed:

1. A device comprising a plurality of colored intracorneal ring segments (ICRS) comprised of at least two outer ring segments and at least two inner ring segments, wherein all at least four ring segments have a common arc length of 160 degrees, an arc width ranging between 800 to 1,000 micron, and wherein the at least two inner rings form an inner diameter ranging between 4.0-4.5 mm and an outer diameter ranging between 6.0-6.5 mm, and the at least two outer rings form an inner diameter ranging between 6.8-7.0 mm and an outer diameter ranging between 8.8-9.0 mm.

2. The device according to claim 1 wherein the outer and inner ring segments are concentric.

3. The device according to claim 1 wherein the ICRS are composed of surgical grade material.

4. The device according to claim 1 wherein each of the colored inner and outer ring segment comprise a plurality of fenestrations.

5. The device according to claim 1 for use in a method for changing an eye color, comprising:
forming a pair of concentric intracorneal tunnels in a mid-periphery of a cornea, wherein the pair of concentric intracorneal tunnels comprises:
an inner tunnel configured to accommodate a pair of colored inner ring segments, wherein each of the colored inner ring segments comprises a plurality of first fenestrations:
an outer tunnel configured to accommodate a pair of colored outer ring segments, wherein each of the pair of outer ring segment comprises a plurality of second fenestrations; and
a cross-section of each the colored ring segments is bilamellar.

6. The device for use according to claim 5, wherein the ICRS are implanted at a depth of between 400 to 430 microns.

7. The device for use according to claim 5, wherein said method is reversible.
